(19) United States
(12) Patent Application Publication
(10) Pub. No.: US 2016/0207970 A1
(43) Pub. Date: Jul. 21, 2016

(54) POLYPEPTIDE FOR THE PROTECTION AGAINST HEART ISCHEMIA-REPERFUSION INJURY

(71) Applicants: Pharis Biotech GmbH, Hannover (DE); University of Copenhagen, Copenhagen K (DK); Rigshospitalet RH, Copenhagen (DK)

(72) Inventors: Marek Treiman, Copenhagen K (DK); Henrik K. Salling, Copenhagen K (DK); Klaus Döhler, Hannover (DE); Thomas Engstrom, Copenhagen (DK)

(21) Appl. No.: 15/088,571

(22) Filed: Apr. 1, 2016

Related U.S. Application Data
(63) Continuation of application No. 14/354,049, filed on Apr. 24, 2014, now abandoned.
(60) Provisional application No. 61/552,721, filed on Oct. 28, 2011.

(30) Foreign Application Priority Data
Oct. 28, 2011 (EP) 11187064.8

(51) Int. Cl. C07K 14/435

(52) U.S. Cl. CPC C07K 14/435; A61K 38/00

(57) ABSTRACT

A peptide of the formula

R¹—NH-HAEGTFTSDVSSYLEGQAAKEFIAWLVK-CONR²R³
wherein R═H or an organic compound comprising from 1 to 10 carbon atoms and R²R³=independently H or an alkyl group of 1 to 4 carbon atoms;
can be used for the treatment and prophylaxis of heart ischemia-reperfusion injury.

### CROSS REFERENCE TO RELATED APPLICATIONS

This application is a continuation of U.S. patent application Ser. No. 14/354,049 filed Apr. 24, 2014, which is a national stage filing of PCT Application No. PCT/EP2012/071366 filed Oct. 29, 2012, which claims priority to European Patent Application No. 11187064.8 filed Oct. 28, 2011, and U.S. Provisional Application No. 61/552,721, filed Oct. 28, 2011, all of which are hereby incorporated herein by reference.

### TECHNICAL FIELD

The invention pertains to a polypeptide for the protection against heart ischemia-reperfusion injury.

### BACKGROUND

Following the treatment for an occlusion of a coronary artery branch (an artery supplying the heart muscle), a damage to heart muscle occurs. This is called reperfusion injury. The phenomenon may be modelled in an experimental setting, in this case on an isolated rat heart.

Ischemia-reperfusion injury (IRI) is a syndrome affecting the myocardium upon blood flow restoration following a sufficiently long interruption, such as encountered in a coronary thrombosis or heart surgery [1,2]. The major components of this syndrome include cardiomyocyte death, myocardial stunning, arrhythmias and no-reflow [1]. Over the last three decades, a large body of experimental research has accumulated aiming to elucidate the pathophysiology of IRI, with a major focus on cardiomyocyte death and approaches to its limitation. One such approach is pharmacological postconditioning, in which a cardioprotective agent is administered coincidentally with flow restoration. This timing makes postconditioning relevant from the clinical perspective, in which limitation of irreversible myocardial damage following a coronary thrombosis and ST-elevation myocardial infarction remains a major objective [3].

Bose, A. K. et al. [4] disclose in Cardiovasc Drugs Ther (2007) 21:253-256, that GLP-1 alone did not decrease myocardial infarction but in combination with the GLP-1 breakdown inhibitor valine pyrrolidide (VP) a significant reduction in myocardial infarction occurred.
EP-A-1 012 188 discloses N—Ac-GLP-1-(7-34)-amide and derivatives thereof for treating diabetes mellitus and obesity.

### SUMMARY OF THE INVENTION

One object of the present invention is to provide a therapy of heart ischemia-reperfusion injury by applying GLP-1 analogues which can be administered as single component and avoiding administration of the drug with a second compound.

The present invention is based on the surprising finding that the peptides of the invention have protective cardiovascular effects without simultaneous administration of other compounds, specifically they have protective effects on the heart against ischemia-reperfusion injury. Postconditioning using N—Ac-GLP-1(7-34)amide N-terminally blocked and C-terminally truncated results in a limitation of ischemia-reperfusion injury in an isolated rat heart. This beneficial effect of N—Ac-GLP-1(7-34)amide was manifested through a diminished diastolic hypercontracture and diminished infarct size.

### BRIEF DESCRIPTION OF DRAWINGS

FIG. 1: This figure outlines the time course for normoxic (A) and ischemia-reperfusion (B) experiments.

FIG. 2: The effects of ischemia-reperfusion on left ventricle diastolic pressure (LVD), left ventricle developed pressure (LVDEV) and rate pressure product (RPP) are shown in FIGS. 2 A-C. In A, * indicates P<0.05 compared to “No peptide” condition.

FIG. 3: Effect of N—Ac-GLP-1(7-34)amide on infarct size. Columns represent mean infarct size (IS) (N=7-14) calculated as percentage of Area at Risk (AAR) (% IS/AAR), with bars indicating SEM. Treatment groups designated as in FIG. 2 are indicated. * indicates P<0.05 compared to “No peptide” condition.

### DETAILED DESCRIPTION

According to the invention a peptide of the formula

(SEQ ID NO: 1)
R¹-NH-HAEGTFTSDVSSYLEGQAAKEFIAWLVK-CONR²R³

wherein R═H or an organic compound comprising from 1 to 10 carbon atoms and R²R³=independently H or an alkyl group of 1 to 4 carbon atoms;

can be used for the treatment and prophylaxis of heart ischemia-reperfusion injury.

In the sequence of the peptides the amino acids are symbolized in the single letter code, but with explicit designating the N-terminal end (R¹—NH—) and C-terminal end (—CONR²R³).

In the peptide for the use of the invention R¹ represents an acyl group.

R¹ is in particular formyl, acetyl, propionyl, isopropionyl and/or R², R³ is independently in particular hydrogen, methyl, ethyl, propyl, isopropyl, n-butyl, sec-butyl, tert-butyl, particularly R²═R³ hydrogen, methyl or ethyl. For example the C-terminal of the peptide may be an amide, dimethylamide, diethyl amide but also mixed amides like mono methyl amide, mono ethylamide, methyl ethylamide and so on. These combinations are easily understood by the skilled person.

The peptide may be modified, wherein A in position 8 of R-GLP-1-(7-34)-amide is substituted by a neutral amino acid selected from the group, consisting of S, S†, G, C†, Sar, beta-ala and Aib; and/or

G in position 10 of R-GLP-1-(7-34)-amide is substituted by a neutral amino acid; and/or

D in position 15 of R-GLP-1-(7-34)-amide is substituted by an acidic amino acid.

In particular in the peptide A may be substituted by S, S†, G, C, C†, Sar, beta-ala and Aib.

The peptide may be formulated in combination with a suitable pharmaceutically acceptable carrier. Such carriers are well known to the skilled person and can be readily derived from textbooks of pharmaceutical formulation.

For example the peptide according to the invention may be formulated in a permanent or pulsative release formulation or formulated for subcutaneous, intravenous, intraarterial, peroral, intramuscular or transpulmonary administration.

As a representative of the peptides of the invention N—Ac-GLP-1-(7-34)-amide, a synthetic analogue of a natural hormone GLP-1, has been shown to diminish this heart ischemia-reperfusion injury in an isolated rat heart without the need to be administered together with a substance inhibiting GLP-1 breakdown, such as VP.

The general type of mode of application for the N—Ac-GLP-1-(7-34)-amide effect is called pharmacological post-conditioning. The cellular and molecular details of this type of biological effect are still not fully understood. While not being bound to any hypothesis or theory of modes of action, a probable mechanism of N—Ac-GLP-1-(7-34)-amide action may involve activation of GLP-1 receptors on heart muscle cells, which in turn activate a number of signalling mechanisms inside the cells, increasing their ability to survive.

The present invention is further illustrated by the following example which, however, is not to be construed as limiting the scope of protection. The features disclosed in the foregoing description and in the following example may, both separately and in any combination thereof, be material for realizing the invention in diverse forms thereof.

### Example

### Method

Global ischemia (35 min)-reperfusion (120 min) was applied in isolated, retrogradely perfused rat hearts. Peptides were present for 15 min at the onset of reperfusion. Cardiac function parameters (beats per minute, left ventricle developed and diastolic pressures, rate-pressure product) throughout the course of the perfusion were measured. Infarct size was determined on collected hearts by 2,3,5-tripehyltetrazolium chloride staining and planimetry.

Results.

N—Ac-GLP-1(7-34)-amide reduced infarct size from 28.4% (SEM 2.8, N=14) to 11.4% (SEM 3.2, N=8; P<0.05). N—Ac-GLP-1(7-34)-amide diminished significantly the post-ischemic diastolic contracture. N—Ac-GLP-1(7-34)-amide had no significant effect on left ventricle developed pressure (LVDEV) or rate-pressure product (RPP), nor did it affect any of the functional heart parameters when administered with no preceding ischemia. The effects of N—Ac-GLP-1(7-34)-amide were abolished by co-administration of a GLP-1 receptor antagonist exendin(9-39).

Materials and Methods

Chemicals.

N—Ac-GLP-1(7-34)-amide was synthetized by Polypeptide Laboratories (San Diego, USA). Exendin (9-39) was purchased from Bachem AG (Switzerland).

Animals and Experimental Procedure.

Male Sprague Dawley rats (330 to 370 g, Taconic, Denmark) were used. The animal studies conformed with the Guide for Care and Use of Laboratory Animals (National Institutes of Health Publication No. 85-23, revised 1996) and Danish legislation governing animal experimentation, 1987, and were carried out after permission had been granted by the Animal Experiments Inspectorate, Ministry of Justice, Denmark.

For anesthesia, a mixture of midazolam (2.5 mg/kg), fluanisone (2.5 mg/kg) and fentanyl citrate (0.08 mg/kg) was administered subcutaneously. Heparin (1000 i.e. per kg) was administered through the femoral vein. The animals were ventilated via a tracheotomy with a mixture of 35% O₂/65% N₂ and the chest cavity was opened. The excised heart was immediately placed in an ice-cold Krebs Henseleit buffer. The heart was quickly mounted onto the Langendorff perfusion system (ADInstruments, UK) and perfused with modified Krebs-Henseleit solution (NaCl 118.5 mM, KCl 4.7 mM, NaHCO₃ 25.0 mM, MgSO₄ 1.2 mM, CaCl₂ 1.4 mM, glucose 11.1 mM), equilibrated to pH 7.4 with a gas mixture of 5% CO₂/95% 02, at 37° C. The left auricle was removed and a size 7 balloon (ADInstruments) was inserted into the left ventricle through the left atrium and adjusted to a diastolic pressure of 4-10 mmHg. Perfusion pressure was set to 70 mm Hg and maintained at this average value by a servo control system (ML175 STH Pump Controller, ADInstruments) continually adjusting the peristaltic pump revolutions according to flow resistance. The hearts were allowed to stabilize for 20 min prior to recording of left ventricle functional parameter baseline values over the next 10 min: pressure (systolic, LVS; diastolic, LVD; developed, LVDEV=LVS-LVD), beats per minute (BPM) and rate-pressure product (RPP). Power Lab 8/30 system and Chart 5 Pro Software from ADInstruments were used for these recordings.

Exclusion Criteria.

Hearts were excluded if average values for the last 10 min of the stabilization period failed to meet the following criteria: BPM: 210-350 min⁻¹, LVDEV: 80-150 mm Hg, RPP: >22,000 (mm Hg×min⁻¹). Hearts were also excluded if ventricular fibrillation lasting more than 5 min occurred during reperfusion.

Treatment Groups.

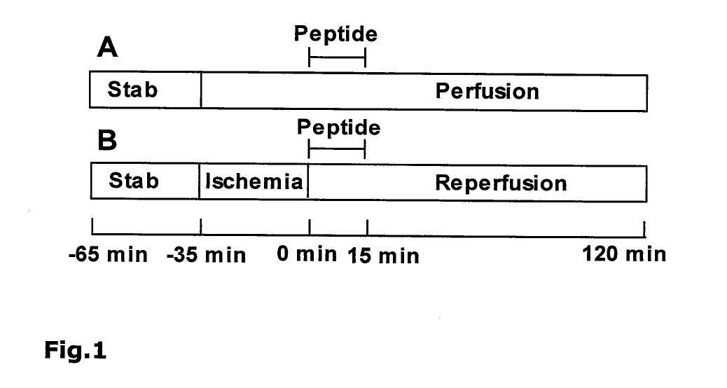

FIG. 1 outlines the time course for normoxic (A) and ischemia-reperfusion (B) experiments. The experimental groups were: control normoxia, no peptide addition; normoxia, N—Ac-GLP-1(7-34)amide 0.3 nM; control ischemia-reperfusion (IR), no peptide addition; IR, N—Ac-GLP-1(7-34)amide 0.3 nM; IR, N—Ac-GLP-1(7-34)amide 0.3 nM+exendin(9-39) 3 nM; IR, exendin(9-39) 3 nM. FIG. 1. shows a scheme illustrating perfusion periods for normoxic perfusion (A) and ischemia-reperfusion (B). “Stab” indicates 30 min stabilization period. When present, peptides were added for 15 min, from the beginning of the last 120 min of perfusion (A) or reperfusion (B). Total perfusion time was always 185 min, consisting of 30 min stabilization, followed by 155 min of normoxic perfusion (FIG. 1A) or 35 min global ischemia-120 min reperfusion (FIG. 1B).

Determination of Infarct Size.

Following reperfusion, the hearts were processed for 2, 3, 5-triphenyltetrazolium chloride staining and planimetric infarct size determination, as described earlier [5]. Quantitation was done by an investigator blinded to the experimental conditions. Infarct size (IS) was expressed as a percentage of total ischemic area at risk (AAR) (% IS/AAR).

Statistical Analysis.

All values are presented as means, with SEM given in parantheses. One-way ANOVA with Dunnett's post hoc test (GraphPad Prism® 5) was used to compare treatment results to control conditions. P<0.05 was considered significant.

Results

Functional Parameters.

Baseline parameter values were (N=14 in all cases): BPM 291 (7) min⁻¹, LVDEV 105.2 (5.4) mmHg, LVD 8.3 (0.7) mmHg, RPP 30302 (1434) mmHg min⁻¹. At the end of normoxic perfusion, the values were (N=5): BPM 215 (14) min⁻¹, LVDEV 75.2 (8.6) mmHg, LVD 19.2 (5.2) mmHg and RPP 16060 (1740) mmHg min⁻¹. The time profiles of these parameters in normoxic experiments were not affected by the presence of N—Ac-GLP-1(7-34)amide between 35 and 50 min of perfusion, i.e. during the period corresponding to peptide administration in the ischemia-reperfusion experiments.

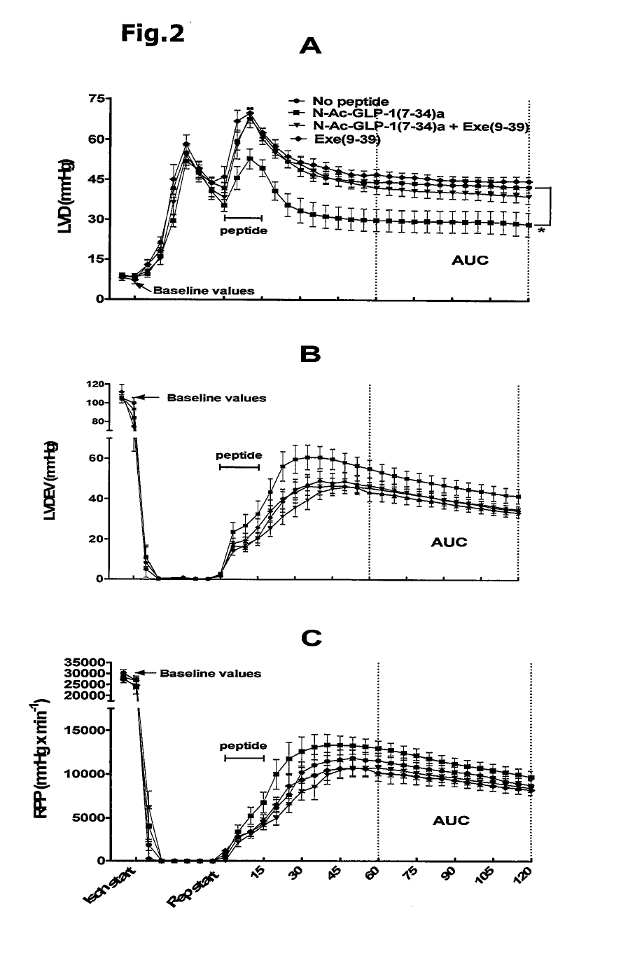

The effects of ischemia-reperfusion on LVD, LVDEV and RPP are shown in FIGS. 2 A-C. Time courses of Left Ventricle Diastolic Pressure (LVD) (A), Left Ventricle Developed Pressure (LVDEV) (B) and Rate-Pressure Product (RPP) (C) in the ischemia-reperfusion experiments. Points represent means of 7-14 experiments, with bars indicating SEM. Start of the ischemia and reperfusion periods, the period of peptide administration (15 min at the onset of reperfusion), and the period for which Area Under the Curve (AUC) was calculated are indicated. Following treatment groups are indicated by symbols (same symbols in A-C): Control ischemia—no peptide present; N—Ac-GLP-1(7-34)amide 0.3 nM; N—Ac-GLP-1(7-34)amide 0.3 nM+Exe(9-39) (exendin(9-39)) 3 nM; Exe(9-39) 3 nM. Points marked “Baseline values” represent means for each group during the last 10 min of the stabilization period. * indicates P<0.05 compared to “No peptide” condition.

LVD rose sharply after flow interruption, declining somewhat towards the end of ischemic period, and rising sharply again at the onset of reperfusion (FIG. 2A). Peak values were reached some 5-10 min after reperfusion start, declining to a near-plateau approximately 60 min later. Area Under the Curve (AUC) was used as a time-integrated measure of functional parameter values over the last 60 min of reperfusion. These AUC-values for LVD were significantly decreased compared to control ischemia, following postconditioning with N—Ac-GLP-1-(7-34)amide; they were not affected either by postconditioning with N—Ac-GLP-1-(7-34)amide in the presence of GLP-1 receptor antagonist exendin(9-39) or when using exendin(9-39) alone (FIG. 2A).

Postconditioning with N—Ac-GLP-1-(7-34)amide did not increase AUC values for LVDEV or RPP significantly (FIGS. 2B and 2C, respectively). A trend towards an LVDEV increase may have been apparent.

Infarct Size.

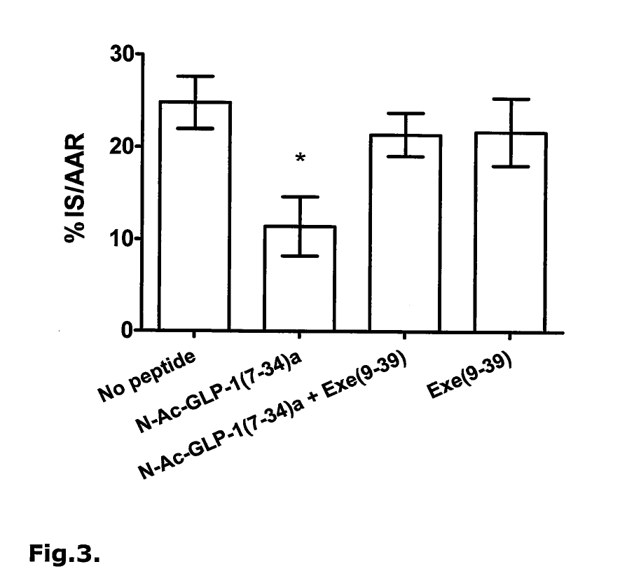

In the absence of postconditioning (control ischemia-reperfusion), infarct size was 24.8% (2.8%, N=14) (FIG. 3). This figure shows also the effect of postconditioning with N—Ac-GLP-1(7-34) amide 0.3 nM on infarct size (% IS/AAR). Treatment groups are designated as in FIG. 2. * indicates P<0.05 compared to “No peptide” condition. Postconditioning with N—Ac-GLP-1-(7-34)amide 0.3 nM reduced the infarct size to 11.4% (3.2%, N=8, P<0.05). Exendin(9-39) has been shown to abolish infarct-limiting actions of GLP-1. Postconditioning with N—Ac-GLP-1(7-34)amide in the presence of exendin(9-39) resulted in infarct size 21.4% (2.4, N=8), not different from control ischemia or from the value in the presence of exendin(9-39) alone (21.7%; 3.6, N=9).

The present invention discloses a cardioprotective effect of the peptides of the invention exemplified by N—Ac-GLP-1(7-34)amide. N—Ac-GLP-1(7-34) amide is a N-terminally acetylated, C-terminally truncated analogue of GLP-1.

N—Ac-GLP-1(7-34)amide was tested for its cardioprotective action as a postconditioning agent. N—Ac-GLP-1(7-34)amide was administered for 15 min immediately following the end of a global ischemia, with reperfusion lasting 120 min. In this mode, N—Ac-GLP-1(7-34)amide had a beneficial effect both at the level of myocardial performance and infarct size. LVD was the parameter affected most strongly, showing a significant decrease following N—Ac-GLP-1(7-34)amide postconditioning (FIG. 2A). A postischemic LVD increase, or hypercontracture, is one of the chief mechanisms contributing to cardiomyocyte death at reperfusion through a sarcolemmal rupture due to a mechanical stress [6]. The LVD-lowering effect of N—Ac-GLP-1(7-34)amide was blocked in the presence of exendin(9-39), a GLP-1 receptor antagonist. Thus, the ameliorating action of N—Ac-GLP-1(7-34)amide on the postischemic contracture was mediated by GLP-1 receptors, known to be present in the myocardium [7].

Diastolic hypercontracture may reflect a poor recovery of ATP synthesis and/or an abnormal Ca²⁺ cycling in recovering myocytes [8]. N—Ac-GLP-1(7-34)amide postconditioning did not affect LVDEV or RPP in a statistically significant manner (FIG. 2B). However, a trend towards LVDEV improvement appeared to exist following postconditioning with N—Ac-GLP-1(7-34)amide, and was abolished in the presence of exendin(9-39) (FIG. 2B).

No effect was found of N—Ac-GLP-1-(7-34)amide on myocardial performance during perfusion under normoxic conditions.

N—Ac-GLP-1(7-34)amide postconditioning caused a significant decrease in the infarct size (FIG. 3), a relative decrease of approximately 54%. Consistent with the effect on LVD discussed above, this infarct size-limiting action of N—Ac-GLP-1(7-34)amide was abolished in the presence of GLP-1 receptor antagonist exendin(9-39).

### REFERENCES

[1] Yellon D M, Hausenloy D J (2007) Myocardial reperfusion injury. N. Engl. J Med. 357:1121-1135.
[2] Turer A T, Hill J A (2010) Pathogenesis of myocardial ischemia-reperfusion injury and rationale for therapy. Am J Cardiol 106:360-368.
[3] Ovize M, Baxter G F, Di Lisa F, et al (2010) Postconditioning and protection from reperfusion injury: where do we stand? Position paper from the working group of cellular biology of the heart of the European Society of Cardiology. Cardiovasc Res 87:406-423.
[4] Bose A. K., Mocanu M M, Carr R D, Yellon D M (2007) Myocardial ischaemia-reperfusion injury is attenuated by intact glucagon like peptide-1 (GLP-1) in the in vitro rat heart and may involve the p70s6K pathway. Cardiovasc Drugs Ther 21:253-256.
[5] Ossum A, van Deurs U, Engstrom T, Jensen J S, Treiman M. (2009) The cardioprotective and inotropic components of the postconditioning effects of GLP-1 and GLP-1(9-36)a in an isolated rat heart. Pharmacol Res 60:411-417.
[6] Inserte J, Barrabes J A, Hernando V, Garcia-Dorado D (2009) Orphan targets for reperfusion injury. Cardiovasc. Res. 83:169-178.
[7] Ban K, Noyan-Ashraf M H, Hoefer J, Bolz S S, Drucker D J, Husain M (2008) Cardioprotective and vasodilatory actions of Glucagon-like Peptide 1 are mediated through both Glucagon-Like Peptide 1 receptor-dependent and -independent pathways. Circulation 117:2340-2350.
[8] Ladilov Y, Efe O, Schafer C, et al (2003) Reoxygenation-induced rigor-type contracture. J Mol Cell Cardiol. 35:1481-1490.

1. A peptide of the formula
(SEQ ID NO: 1)
R¹-NH-HAEGTFTSDVSSYLEGQAAKEFIAWLVK-CONR²R³
wherein R═H or an organic compound comprising from 1 to 10 carbon atoms and R²R³=independently H or an alkyl group of 1 to 4 carbon atoms.

2. The peptide of claim 1, wherein R¹ represents an acyl group.

3. The peptide of claim 1, wherein R¹ is formyl, acetyl, propionyl, isopropionyl and/or R², R³ is independently hydrogen, methyl, ethyl, propyl, isopropyl, n-butyl, sec-butyl, tert-butyl, in particular R²═R³ hydrogen, methyl or ethyl.

4. The peptide according to claim 1, wherein
A in position 8 of R-GLP-1-(7-34)-amide is substituted by a neutral amino acid selected from the group consisting of S, S†, G, C, C†, Sar, beta-ala and Aib; and/or
G in position 10 of R-GLP-1-(7-34)-amide is substituted by a neutral amino acid; and/or
D in position 15 of R-GLP-1-(7-34)-amide is substituted by an acidic amino acid.

5. The peptide according to claim 4 wherein A is substituted by an amino acid selected from the group consisting of S, S†, G, C, C†, Sar, beta-ala and Aib.

6. The peptide of claim 1 in combination with a suitable pharmaceutically acceptable carrier.

7. The peptide of claim 1 characterized in that said peptide is formulated in a permanent or pulsative release.

8. The peptide of claim 1 characterized in that said peptide is formulated for subcutaneous, intravenous, intraarterial, peroral, intramuscular or transpulmonary administration.

9. A method of using a peptide comprising:
administering the peptide to a patient for treatment or prophylaxis of heart ischemia-reperfusion injury
wherein the peptide is of the formula
(SEQ ID No: 1)
R¹-NH-HAEGTFTSDVSSYLEGQAAKEFIAWLVK-CONR²R³
wherein R═H or an organic compound comprising from 1 to 10 carbon atoms and R²R³=independently H or an alkyl group of 1 to 4 carbon atoms.

10. The method of claim 9 wherein the administration is selected from the group consisting of subcutaneous, intravenous, intraarterial, peroral, intramuscular, and transpulmonary administration.

11. The method of claim 9 wherein the administration comprises permanent or pulsative Release of the peptide.

12. The method of claim 9 wherein R¹ represents an acyl group.

13. The method of claim 9 wherein R¹ is formyl, acetyl, propionyl, isopropionyl and/or R², R³ is independently hydrogen, methyl, ethyl, propyl, isopropyl, n-butyl, sec-butyl, tert-butyl, in particular R²═R³ hydrogen, methyl or ethyl.

14. The method of claim 9, wherein R¹ represents an acyl group.

15. The method of claim 9, wherein R¹ is formyl, acetyl, propionyl, isopropionyl and/or R², R³ is independently hydrogen, methyl, ethyl, propyl, isopropyl, n-butyl, sec-butyl, tert-butyl, in particular R²═R³ hydrogen, methyl or ethyl.

16. The method according to claim 9, wherein
A in position 8 of R-GLP-1-(7-34)-amide is substituted by a neutral amino acid selected from the group consisting of S, S†, G, C, C†, Sar, beta-ala and Aib; and/or
G in position 10 of R-GLP-1-(7-34)-amide is substituted by a neutral amino acid; and/or
D in position 15 of R-GLP-1-(7-34)-amide is substituted by an acidic amino acid.

17. The method according to claim 16 wherein A is substituted by an amino acid selected from the group consisting of S, S†, G, C, C†, Sar, beta-ala and Aib.

18. The method of claim 9 wherein the peptide is in combination with a suitable pharmaceutically acceptable carrier.

19. The method of claim 9 wherein the peptide is characterized in that said peptide is formulated in a permanent or pulsative release.

20. The method of claim 9 wherein the peptide is formulated for subcutaneous, intravenous, intraarterial, peroral, intramuscular or transpulmonary administration.


SEQUENCE LISTING (1 sequence)

SEQ ID NO: 1 - protein, 28 aa, human
HAEGTFTSDVSSYLEGQAAKEFIAWLVK